(19) United States
(12) Patent Application Publication
(10) Pub. No.: US 2011/0112128 A1
(43) Pub. Date: May 12, 2011

(54) METHOD OF TREATING ILEUS BY PHARMACOLOGICAL ACTIVATION OF CHOLINERGIC RECEPTORS

(76) Inventors: Kevin J. Tracey, Old Greenwich, CT (US); Mitchell P. Fink, Pittsburgh, PA (US)

(21) Appl. No.: 12/839,571

(22) Filed: Jul. 20, 2010

Related U.S. Application Data
(63) Continuation of application No. 11/645,120, filed on Dec. 22, 2006, now abandoned.
Application No. 11/645,120 is a continuation of application No. PCT/US05/22495, filed on Jun. 23, 2005.
(60) Provisional application No. 60/582,545, filed on Jun. 23, 2004.

(51) Int. Cl. A61K 31/439; A61P 1/00; A61K 31/444; A61K 31/46; A61K 31/14

(52) U.S. Cl. 514/278; 514/334; 514/305; 514/304; 514/642

(57) ABSTRACT

A method of treating ileus in a subject by administering to the subject an effective amount of a pharmacological agent that increases the activity of cholinergic receptor in a subject. Examples of pharmacological agents are brain muscarinic agonist, cholinergic agonist or cholinesterase inhibitor. The methods of the present invention can be used to treat ileus caused by abdominal surgery, or administration of narcotics or chemotherapeutic agents such as during cancer chemotherapy.

### RELATED APPLICATION

This application is a continuation of U.S. patent application Ser. No. 11/645,120, filed on Dec. 22, 2006, which is a continuation of International Application No. PCT/US2005/022495, which designated the United States and was filed on Jun. 23, 2005, published in English, which claims the benefit of U.S. Provisional Application No. 60/582,545, filed on Jun. 23, 2004. The entire teachings of the above applications are incorporated herein by reference.

### BACKGROUND OF THE INVENTION

Ileus is a partial or complete non-mechanical obstruction of the entire gastrointestinal tract, including an obstruction of the small and/or large intestine. Ileus occurs when peristalsis, the rhythmic contraction that moves material through the bowel, stops. Ileus can be caused, for example, by manipulation of the intestines during abdominal surgery, or administration of narcotics or chemotherapeutic agents.

While postoperative ileus usually resolves spontaneously within about 36 to 96 hours, until it resolves, supervised bed rest and bowel rest in a hospital is the current therapy. Patients with ileus take no food or medications by mouth. The patients are hydrated intravenously and gastric decompression is provided through the use of a nasogastric tube. The discomfort, inconvenience and economic costs of this current therapy are substantial.

Methods for preventing ileus are presently unavailable, and methods for treating ileus are currently inadequate. Thus, there is an urgent need for new methods of preventing and/or ameliorating the effects of ileus.

### SUMMARY OF THE INVENTION

It has now been discovered that ileus can be treated in a subject by administering certain pharmaceutical agents that increase the activity of cholinergic receptors.

Accordingly, in one embodiment, the present invention is a method of treating ileus in a subject, comprising administering to the subject a pharmacological agent which increases the cholinergic receptor activity.

In another embodiment, the present invention is a method of treating ileus in a subject, comprising administering an effective amount of a muscarinic agonist to the subject.

In another embodiment, the present invention is a method of treating ileus in a subject, comprising administering an effective amount of a cholinergic agonist to the subject. In another embodiment, the cholinergic agonist is selective for an α7 nicotinic receptor.

### DETAILED DESCRIPTION OF THE INVENTION

The present invention is based on the discovery that ileus can be treated in a subject by administering to the subject a pharmaceutical agent that increases the activity of cholinergic receptors. An agent which “increases cholinergic receptor activity,” includes both direct and indirect pharmacological activation of the receptor. Direct pharmacological activation includes agonists, i.e., compounds which bind and stimulate the receptor. Indirect pharmacological activation refers to activation of the receptor other than by binding. Examples include agents which activate the vagus nerve, thereby resulting in release of acetyl choline from the terminus (e.g., brain muscarinic receptor agents agonists). Such increase can be achieved, in one embodiment, by administration of acetylcholine receptor agonists. In yet another embodiment, cholinergic receptor activity is increased by stimulating vagus nerve by pharmacological means.

The vagus nerve enervates principal organs including, the pharynx, the larynx, the esophagus, the heart, the lungs, the stomach, the pancreas, the spleen, the kidneys, the adrenal glands, the small and large intestine, the colon, and the liver. As used herein, the vagus nerve includes nerves that branch off from the main vagus nerve, as well as ganglions or postganglionic neurons that are connected to the vagus nerve.

As used herein, a subject is preferably a mammal, more preferably a human patient but can also be a companion animal (e.g., dog or cat), a farm animal (e.g., horse, cow, or sheep) or a laboratory animal (e.g., rat, mouse, or guinea pig).

As used herein, “ileus” means the arrest (stoppage or decreased activity) of intestinal peristalsis having causes other than interruption of blood flow to the intestines or by reperfusion in the intestines or neurological damage. Ileus can be caused, for example, by manipulation of the intestines during abdominal surgery, or administration of narcotics, for example, morphine sulfate, meperidine hydrochloride, codeine phosphate, or oxycodone hydrochloride, or chemotherapeutic agents such as vincristine, vinorelbine tartrate, doxorubicin hydrochloride or BCNU (carmustine). Accordingly, in one embodiment, ileus is an acute post-operative ileus. In another embodiment, ileus is caused by administration of narcotics. Ileus can be detected, for example, by auscultation. Symptoms of ileus include, but are not limited to abdominal distention, vomiting, constipation, cramps, hiccups, or gaseous distention of isolated segments of small and/or large bowel or colon, as detected by X-rays, computed tomography scans or ultrasound.

### Brain Muscarinic Receptors Agonists

In one embodiment of the present invention, the pharmacological agent is an agonist that activates a muscarinic receptor in the brain (such as a muscarinic agonist). As used herein, a muscarinic agonist is a compound that can bind to and activate a muscarinic receptor to produce a desired physiological effect, here, alleviation of the ileus symptoms. A muscarinic receptor is a cholinergic receptor which contains a recognition site for a muscarinic agonist (such as muscarine). In one embodiment, the muscarinic agonist is non-selective and acts on other receptors in addition to muscarinic receptors, for example, another cholinergic receptor. An example of such a muscarinic agonist is acetylcholine. In a preferred embodiment, the muscarinic agonist activates muscarinic receptors to a greater extent than other cholinergic receptors, for example, nicotinic receptors (for example at least 10% greater, 20% greater, 50% greater, 75% greater, 90% greater, or 95% greater).

In a preferred embodiment the muscarinic agonist is selective for an M1, M2, or M4 muscarinic receptor (as disclosed in U.S. Pat. No. 6,602,891, U.S. Pat. No. 6,528,529, U.S. Pat. No. 5,726,179, U.S. Pat. No. 5,718,912, U.S. Pat. No. 5,618,818, U.S. Pat. No. 5,403,845, U.S. Pat. No. 5,175,166, U.S. Pat. No. 5,106,853, U.S. Pat. No. 5,073,560 and U.S. patent application Ser. No. 10/375,696 filed Feb. 26, 2003, the contents of each of which are incorporated herein by reference in their entirety). As used herein, an agonist that is selective for an M1, M2, or M4 receptor is an agonist that activates an M1, M2, and/or M4 receptor to a greater extent than at least one, or at least two, or at least five other muscarinic receptor subtypes (for example, M3 or M5 muscarinic receptors) and/or at least one, or at least two, or at least five other cholinergic receptors. In a preferred embodiment, the agonist has at least 10% greater activation activity, 20% greater activation activity, 50% greater activation activity, 75% greater activation activity, 90% greater activation activity, or 95% greater activation activity than with respect to muscarinic and/or cholinergic receptor subtypes other than M1, M2, and/or M4 receptors. Activation activity can be determined using assays known to one of skill in the art.

Nonlimiting examples of preferred muscarinic agonists useful for these methods include: muscarine, McN-A-343, and MT-3. In a most preferred embodiment, the muscarinic agonist is N,N′-bis(3,5-diacetylphenyl) decanediamide tetrakis (amidinohydrazone) tetrahydrochloride (CNI-1493), which has the following structural formula:

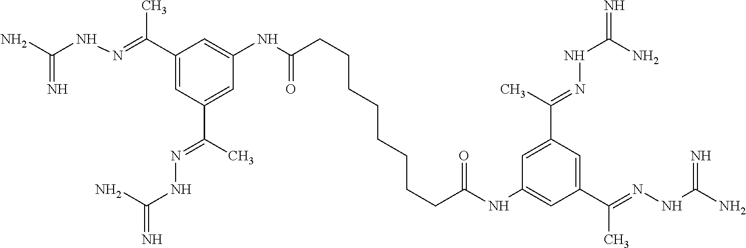

In another embodiment, the muscarinic agonist is a CNI-1493 compound. As used herein, a CNI-1493 compound is an aromatic guanylhydrazone (more properly termed amidinohydrazone, i.e., NH₂(CNH)—NH—N═), for example, a compound having the structural formula I:

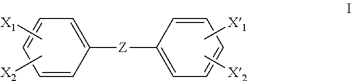

X₂ is NH₂(CNH)—NH—N═CH—, NH₂(CNH)—NH—N═CCH₃—, or H—; X₁, X′₁ and X′₂ independently are NH₂(CNH)—NH—N═CH— or NH₂(CNH)—NH—N═CCH₃—; Z is —NH(CO)NH—, —(C₆H₄)—, —(C₅NH₃)—, or -A-(CH₂)ₙ-A-, n is 2-10, which is unsubstituted, mono- or di-C-methyl substituted, or a mono or di-unsaturated derivative thereof; and A, independently, is —NH(CO)—, —NH(CO)NH—, —NH—, or —O—, and pharmaceutically acceptable salts thereof. A preferred embodiment includes those compounds where A is a single functionality. Also included are compounds having the structural formula I when X₁ and X₂ are H; X′₁ and X′₂ independently are NH₂(CNH)—NH—N═CH— or NH₂(CNH)—NH—N═CCH₃—; Z is -A-(CH₂)ₙ-A-, n is 3-8; A is —NH(CO)— or —NH(CO)NH—; and pharmaceutically acceptable salts thereof. Also included are compounds of structural formula I when X₁ and X₂ are H; X′₁ and X′₂ independently are NH₂(CNH)—NH—N═CH— or NH₂(CNH)—NH—N═CCH₃—; Z is —O—(CH₂)₂—O—; and pharmaceutically acceptable salts thereof.

Further examples of CNI-1493 compounds include compounds of structural formula I when X₂ is NH₂(CNH)—NH—N═CH—, NH₂(CNH)—NH—N═CCH₃— or H—; X₁, X′₁ and X′₂ are NH₂(CNH)—NH—N═CH— or NH₂(CNH)—NH—N═CCH₃—; and Z is —O—(CH₂)ₙ—O—, n is 2-10; pharmaceutically acceptable salts thereof; and the related genus, when X₂ is other than H, X₂ is meta or para to X₁ and when, X′₂ is meta or para to X′₁. Another embodiment includes a compound having structural formula I when X₂ is NH₂(CNH)—NH—N═CH—, NH₂(CNH)—NH—N═CCH₃—, or H; X₁, X′₁ and X′₂, are NH₂(CNH)—NH—N═CH— or NH₂(CNH)—NH—N═CCH₃—; Z is —NH—(C═O)—NH—; pharmaceutically acceptable salts thereof; and the related genus when X₂ is other than H, X₂ is meta or para to X₁ and when X′₂ is meta or para to X′₁.

A CNI-1493 compound also includes an aromatic guanylhydrazone compound having the structural formula II:

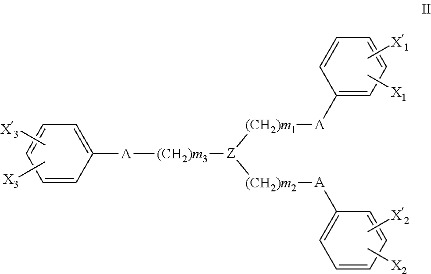

X₁, X₂, and X₃ independently are NH₂(CNH)—NH—N═CH— or NH₂(CNH)—NH—N═CCH₃—, X′₁, X′₂, and X′₃ independently are H, NH₂(CNH)—NH—N═CH— or NH₂(CNH)—NH—N═CCH₃—; Z is (C₆H₃), when m₁, m₂, and m₃ are 0 or Z is N, when, independently, m₁, m₂, and m₃ are 2-6, and A is —NH(CO)—, —NH(CO)NH—, —NH—, or —O—; and pharmaceutically acceptable salts thereof. Further examples of compounds of structural formula II include the genus wherein, when any of X′₁, X′₂, and X′₃ are other than H, then the corresponding substituent of the group consisting of X₁, X₂, and X₃ is meta or para to X′₁, X′₂, and X′₃, respectively; the genus when m₁, m₂, and m₃ are 0 and A is —NH(CO)—; and the genus when m₁, m₂, and m₃ are 2-6, A is —NH(CO)NH—, and pharmaceutically acceptable salts thereof. Examples of CNI-1493 compounds and methods for making such compounds are described in U.S. Pat. No. 5,854,289 (the contents of which are incorporated herein by reference).

### Cholinergic Agonists

In one embodiment of the present invention, treatment of ileus comprises administering an effective amount of a cholinergic agonist to a subject, thus treating or alleviating the symptoms of ileus in said subject. As used herein, a cholinergic agonist is a compound that binds to and activates a cholinergic receptor producing a desired physiological effect, here, treatment of ileus or alleviation of symptoms of ileus in a subject. The skilled artisan can determine whether any particular compound is a cholinergic agonist by any of several well known methods. The cholinergic agonist can be administered to the subject or be naturally produced in vivo. Nonlimiting examples of cholinergic agonists suitable for use in the disclosed invention include: acetylcholine, nicotine, muscarine, carbachol, galantamine, arecoline, cevimeline, and levamisole. In one embodiment the cholinergic agonist is acetylcholine, nicotine, or muscarine.

In one embodiment, the cholinergic agonist is an α7 selective nicotinic cholinergic agonist. As used herein an α7 selective nicotinic cholinergic agonist is a compound that selectively binds to and activates an α7 nicotinic cholinergic receptor in a subject. Nicotinic cholinergic receptors are a family of ligand-gated, pentameric ion channels. In humans, 16 different subunits (α1-7, α9-10, β1-4, δ, ε, and γ) have been identified that form a large number of homo- and hetero-pentameric receptors with distinct structural and pharmacological properties (Lindstrom, J. M., Nicotinic Acetylcholine Receptors. In “Hand Book of Receptors and Channels: Ligand- and Voltage-Gated Ion Channels” Edited by R. Alan North CRC Press Inc., (1995); Leonard, S., & Bertrand, D., Neuronal nicotinic receptors: from structure to function. Nicotine & Tobacco Res. 3:203-223 (2001); Le Novere, N., & Changeux, J-P., Molecular evolution of the nicotinic acetylcholine receptor: an example of multigene family in excitable cells, J. Mol. Evol., 40:155-172 (1995)).

As used herein, a cholinergic agonist is selective for an α7 nicotinic cholinergic receptor if that agonist activates an α7 nicotinic cholinergic receptor to a greater extent than the agonist activates at least one other nicotinic receptor. It is preferred that the α7 selective nicotinic agonist activates the α7 nicotinic receptor at least two-fold, at least five-fold, at least ten-fold, and most preferably at least fifty-fold more than at least one other nicotinic receptor (and preferably at least two, three, or five other nicotinic receptors). Most preferably, the α7 selective nicotinic agonist will not activate another nicotinic receptor to any measurable degree (i.e., significant at P=0.05 vs. untreated receptor in a well-controlled comparison).

Such an activation difference can be measured by comparing activation of the various receptors by any known method, for example using an in vitro receptor binding assay, such as those produced by NovaScreen Biosciences Corporation (Hanover Md.), or by the methods disclosed in WO 02/44176 (α4β2 tested), U.S. Pat. No. 6,407,095 (peripheral nicotinic receptor of the ganglion type), U.S. Patent Application Publication No. 2002/0086871 (binding of labeled ligand to membranes prepared from GH₄Cl cells transfected with the receptor of interest), and WO 97/30998. References which describe methods of determining agonists that are selective for α7 receptors include: U.S. Pat. No. 5,977,144 (Table 1), WO 02/057275 (pg 41-42), and Holladay et al., Neuronal Nicotinic Acetylcholine Receptors as Targets for Drug Discovery, Journal of Medicinal Chemistry, 40:4169-4194 (1997), the teachings of these references are incorporated herein by reference in their entirety. Assays for other nicotinic receptor subtypes are known to the skilled artisan.

In one embodiment the α7 selective nicotinic agonist is a compound of structural formula III:

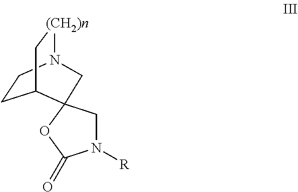

R is hydrogen or methyl, and n is 0 or 1, and pharmaceutically acceptable salts thereof. In a preferred embodiment the α7 selective nicotinic agonist is (−)-Spiro[1-azabicyclo[2.2.2]octane-3,5′-oxazolidin-2′-one]. Methods of preparation of compounds of structural formula III are described in U.S. Pat. No. 5,902,814, the contents of which are incorporated herein by reference in their entirety.

In another embodiment, the α7 selective nicotinic agonist is a compound of structural formula IV:

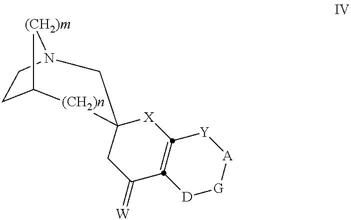

m is 1 or 2; n is 0 or 1; Y is CH, N or NO; X is oxygen or sulfur; W is oxygen, H₂ or F₂; A is N or C(R²); G is N or C(R³); D is N or C(R⁴); with the proviso that no more than one of A, G and D is nitrogen but at least one of Y, A, G, and D is nitrogen or NO; R¹ is hydrogen or C₁ to C₄ alkyl, R², R³, and R⁴ are independently hydrogen, halogen, C₁-C₄ alkyl, C₂-C₄ alkenyl, C₂-C₄ alkynyl, aryl, heteroaryl, OH, OC₁-C₄ alkyl, CO₂R¹, —CN, —NO₂, —NR⁵R⁶, —CF₃, or —OSO₂CF₃, or R² and R³, or R³ and R⁴, respectively, may together form another six membered aromatic or heteroaromatic ring sharing A and G, or G and D, respectively, containing between zero and two nitrogen atoms, and substituted with one to two of the following substitutents: independently hydrogen, halogen, C₁-C₄ alkyl, C₂-C₄ alkenyl, C₂-C₄ alkynyl, aryl, heteroaryl, OH, OC₁-C₄ alkyl, CO₂R¹, —CN, —NO₂, —NR⁵R⁶, —CF₃, or —OSO₂CF₃; R⁵ and R⁶ are independently hydrogen, C₁-C₄ alkyl, C(O)R⁷, C(O)NHR⁸, C(O)OR⁹, SO₂R¹⁰ or may together be (CH₂)ⱼQ(CH₂)ₖ, where Q is O, S, NR¹¹, or a bond; j is 2 to 7; k is 0 to 2; and R⁷, R⁸, R⁹, R¹⁰ and R¹¹ are independently C₁-C₄, alkyl, aryl, or heteroaryl; an enantiomer thereof, or a pharmaceutically acceptable salt thereof. In preferred embodiments, the α7 selective nicotinic agonist is a compound of structural formula IV when m is 2; n is 0; X is oxygen; A is C(R²); G is C(R³); and D is C(R⁴). In a particular preferred embodiment the α7 selective nicotinic agonist is (R)-(−)-5′-phenylspiro[1-aziobicyclo[2.2.2]octane-3,2′(3′H)-furo[2,3-b]pyridine]. Methods of preparation of compounds of structural formula IV are described in the U.S. Pat. No. 6,110,914, the contents of which are incorporated herein by reference in their entirety.

In yet another embodiment the α7 selective nicotinic agonist is a compound of structural formula V:

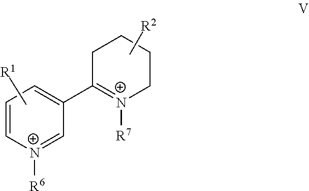

R¹ is hydrogen or C₁-C₄ alkyl, R⁶ and R⁷ are independently selected from hydrogen, or C₁-C₄ alkyl or may be absent; and R² is:

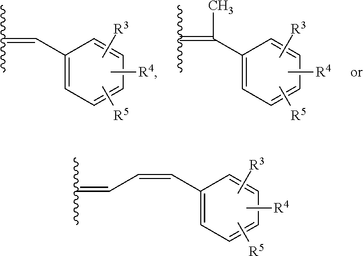

R³, R⁴, and R⁵ are hydrogen, C₁-C₄ alkyl optionally substituted with N,N-dialkylamino having 1 to 4 carbons in each of the alkyls, C₁-C₆ alkoxy optionally substituted with N,N-dialkylamino having 1 to 4 carbons in each of the alkyls, carboalkoxy having 1 to 4 carbons in the alkoxy, amino, amido having 1 to 4 carbons in the acyl, cyano, and N,N-dialkylamino having 1 to 4 carbons in each of the alkyls, halo, hydroxyl or nitro.

In preferred embodiments, the α7 selective nicotinic agonist is a compound of structural formula V when R² is attached to the 3-position of the tetrahydropyridine ring. In another preferred embodiment when R³, which may preferably be attached to the 4- or the 2-position of the phenyl ring, is: amino, hydroxyl, chloro, cyano, dimethylamino, methyl, methoxy, acetylamino, acetoxy, or nitro. In one particular preferred embodiment the α7 selective nicotinic agonist is a compound of structural formula V, when R³ is hydroxyl, and R¹, R⁴, and R⁵ are hydrogen. In another particular preferred embodiment the α7 selective nicotinic agonist is a compound of structural formula V, when R³ is acetylamino and R¹, R⁴, and R⁵ are hydrogen, In another particular preferred embodiment the α7 selective nicotinic agonist is a compound of structural formula V, when R³ is acetoxy and R¹, R⁴, and R⁵ are hydrogen. In another particular preferred embodiment the α7 selective nicotinic agonist is a compound of structural formula V, when R³ is methoxy and R¹, R⁴, and R⁵ are hydrogen. In another particular preferred embodiment the α7 selective nicotinic agonist is a compound of structural formula V, when R³ is methoxy and R¹ and R⁴ are hydrogen, and further when, R³ is attached to the 2-position of the phenyl ring, and R⁵, which is attached to the 4-position of the phenyl ring, is methoxy or hydroxy.

In a preferred embodiment the α7 selective nicotinic agonist is: 3-(2,4-dimethoxybenzylidine)anabaseine (DMXB-A), 3-(4-hydroxybenzylidene)anabaseine, 3-(4-methoxybenzylidene)anabaseine, 3-(4-aminobenzylidene)anabaseine, 3-(4-hydroxy-2-methoxybenzylidene)anabaseine, 3-(4-methoxy-2-hydroxybenzylidene)anabaseine, trans-3-cinnamylidene anabaseine, trans-3-(2-methoxy-cinnamylidene)anabaseine, or trans-3-(4-methoxycinnamylidene)anabaseine.

Methods of preparation of compounds of structural formula V are described in U.S. Pat. Nos. 5,977,144 and 5,741,802, the contents of which are incorporated herein by reference in their entirety.

In further embodiments the α7 selective nicotinic agonist is a compound of structural formula VI:

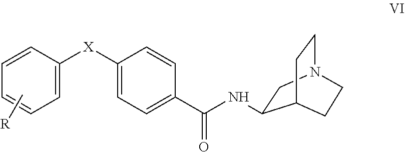

X is O or S; R is H, OR¹, NHC(O)R¹, or a halogen; and R¹ is C₁-C₄ alkyl; or a pharmaceutically acceptable salt thereof. In a particular preferred embodiment the α7 selective nicotinic agonist is:
N-[(3R)-1-azabicyclo[2.2.2]oct-3-yl]-4-(4-hydroxyphenoxy)benzamide,
N-[(3R)-1-azabicyclo[2.2.2]oct-3-yl]-4-(4-acetamidophenoxy)benzamide,
N-[(3R)-1-azabicyclo[2.2.2]oct-3-yl]-4-(phenylsulfanyl)benzamide, or
N-[(3R)-1-azabicyclo[2.2.2]oct-3-yl]-4-(3-chlorophenylsulphonyl)benzamide.

Methods of preparation of compounds with structural formula VI have been described in the U.S. Patent Application 2002/0040035, the contents of which are incorporated herein by reference in their entirety.

In yet another embodiment the α7 selective nicotinic agonist is (1-aza-bicyclo[2.2.2]oct-3-yl)-carbamic acid 1-(2-fluorophenyl)-ethyl ester, Methods of preparation of this compound have been described in the U.S. Patent Application Publication 2002/0040035, the contents of which are incorporated herein by reference in their entirety.

In an even more preferred embodiment the α7 selective nicotinic agonist is: DMXB-A, 3-(4-hydroxy-2-methoxybenzylidene)anabaseine, 3-(4-hydroxy-2-methoxybenzylidene)anabaseine, (R)-(−)-5′-phenylspiro[1-azabicyclo[2.2.2]octane-3,2′octane-3,2′(3′H)-furo[2,3-b]pyridine], (−)-spiro-[1-azabicyclo[2.2.2]octane-3,5′-oxazolidin-2′-one], or cocaine methiodide.

In another preferred embodiment, the α7 selective nicotinic agonist is selected from the group consisting of trans-3-cinnamylidene anabaseine, trans-3-(2-methoxy-cinnamylidene)anabaseine, and trans-3-(4-methoxycinnamylidene)anabaseine.

In yet another embodiment, the α7 selective nicotinic agonist is an antibody which is a selective agonist (most preferably a specific agonist) for the α7 nicotinic receptor. The antibodies can be polyclonal or monoclonal; may be from human, non-human eukaryotic, cellular, fungal or bacterial sources; may be encoded by genomic or vector-borne coding sequences; and may be elicited against native or recombinant α7 or fragments thereof with or without the use of adjuvants, all according to a variety of methods and procedures well-known in the art for generating and producing antibodies. Other examples of such useful antibodies include but are not limited to chimeric, single-chain, and various human or humanized types of antibodies, as well as various fragments thereof such as Fab fragments and fragments produced from specialized expression systems.

In additional embodiments, the α7 selective nicotinic agonist is an aptamer which is a selective agonist (more preferably a specific agonist) for the α7 nicotinic receptor. Aptamers are single stranded oligonucleotides or oligonucleotide analogs that bind to a particular target molecule, such as a protein or a small molecule (e.g., a steroid or a drug, etc.). Thus aptamers are the oligonucleotide analogy to antibodies. However, aptamers are smaller than antibodies, generally in the range of 50-100 nt. Their binding is highly dependent on the secondary structure formed by the aptamer oligonucleotide. Both RNA and single stranded DNA (or analog), aptamers are known. See, e.g., Burke et al., J. Mol. Biol., 264(4): 650-666 (1996); Ellington and Szostak, Nature, 346(6287): 818-822 (1990); Hirao et al., Mol Divers., 4(2): 75-89 (1998); Jaeger et al., The EMBO Journal 17(15): 4535-4542 (1998); Kensch et al., J. Biol. Chem., 275(24): 18271-18278 (2000); Schneider et al., Biochemistry, 34(29): 9599-9610 (1995); and U.S. Pat. Nos. 5,496,938; 5,503,978; 5,580,737; 5,654,151; 5,726,017; 5,773,598; 5,786,462; 6,028,186; 6,110,900; 6,124,449; 6,127,119; 6,140,490; 6,147,204; 6,168,778; and 6,171,795. Aptamers can also be expressed from a transfected vector (Joshi et al., J. Virol., 76(13), 6545-6557 (2002)).

Aptamers that bind to virtually any particular target can be selected by using an iterative process called SELEX, which stands for Systematic Evolution of Ligands by EXponential enrichment (Burke et al., J. Mol. Biol., 264(4): 650-666 (1996); Ellington and Szostak, Nature, 346(6287): 818-822 (1990); Schneider et al., Biochemistry, 34(29): 9599-9610 (1995); Tuerk et al., Proc. Natl. Acad. Sci. USA, 89: 6988-6992 (1992); Tuerk and Gold, Science, 249(4968): 505-510 (1990)). Several variations of SELEX have been developed which improve the process and allow its use under particular circumstances. See, e.g., U.S. Pat. Nos. 5,472,841; 5,503,978; 5,567,588; 5,582,981; 5,637,459; 5,683,867; 5,705,337; 5,712,375; and 6,083,696. Thus, the production of aptamers to any particular oligopeptide, including the α7 nicotinic receptor, requires no undue experimentation.

### Other Pharmaceutical Agents

In another embodiment, treating ileus in a subject comprises administering to the subject an effective amount of a non-steriodal anti-inflammatory drug (NSAID). Examples of suitable NSAIDs include: aspirin, indomethacin, and ibuprofen. Alternatively, ileus is treated by administering to the subject an effective amount of amiodarone or α-melanocyte-stimulating hormone (MSH).

As described above, the compounds can be administered in the form of a pharmaceutically acceptable salt. This includes compounds disclosed herein which possess a sufficiently acidic, a sufficiently basic, or both functional groups, and accordingly can react with any of a number of organic or inorganic bases, and organic or inorganic acids, to form a salt. Acids commonly employed to form acid addition salts from compounds with basic groups, are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfate, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethyl amine, bis-(2-hydroxyethyl)amine, tri-(2-hydroxyethyl)amine, procaine, dibenzylpiperidine, -benzyl-phenethylamine, dehydroabietylamine, N,N′-bisdehydroabietylamine, glucamine, N-methylglucamine, collidine, quinine, quinoline, and basic amino acid such as lysine and arginine. These salts may be prepared by methods known to those skilled in the art.

The term “alkyl”, as used herein, unless otherwise indicated, includes saturated monovalent hydrocarbon radicals having straight or branched moieties, typically C₁-C₁₀, preferably C₁-C₆. Examples of alkyl groups include, but are not limited to, methyl, ethyl, propyl, isopropyl, and t-butyl.

The term “alkenyl”, as used herein, includes alkyl moieties, as defined above, having at least one carbon-carbon double bond. Examples of alkenyl groups include, but are not limited to, ethenyl and propenyl.

The term “alkynyl”, as used herein, includes alkyl moieties, as defined above, having at least one carbon-carbon triple bond. Examples of alkynyl groups include, but are not limited to, ethynyl and 2-propynyl.

The term “alkoxy”, as used herein, means an “alkyl-O—” group, wherein alkyl is defined above.

The term “cycloalkyl”, as used herein, includes non-aromatic saturated cyclic alkyl moieties, wherein alkyl is as defined above. Examples of cycloalkyl include, but are not limited to, cyclopropyl, cyclobutyl, cyclopentyl, cyclohexyl, and cycloheptyl. “Bicycloalkyl” groups are non-aromatic saturated carbocyclic groups consisting of two rings. Examples of bicycloalkyl groups include, but are not limited to, bicyclo-[2.2.2]-octyl and norbornyl. The term “cycloalkenyl” and “bicycloalkenyl” refer to non-aromatic carbocyclic, cycloalkyl, and bicycloalkyl moieties as defined above, except comprising of one or more carbon-carbon double bonds connecting carbon ring members (an “endocyclic” double bond) and/or one or more carbon-carbon double bonds connecting a carbon ring member and an adjacent non-ring carbon (an “exocyclic” double bond). Examples of cycloalkenyl groups include, but are not limited to, cyclopentenyl and cyclohexenyl. A non-limiting example of a bicycloalkenyl group is norborenyl. Cycloalkyl, cycloalkenyl, bicycloalkyl, and bicycloalkenyl groups also include groups similar to those described above for each of these respective categories, but which are substituted with one or more oxo moieties. Examples of such groups with oxo moieties include, but are not limited to, oxocyclopentyl, oxocyclobutyl, ococyclopentenyl, and norcamphoryl.

The term “cycloalkoxy”, as used herein, includes “cycloalkyl-O—” group, wherein cycloalkyl is defined above.

The term “aryl”, as used herein, refers to carbocyclic group. Examples of aryl groups include, but are not limited to, phenyl and naphthyl.

The term “heteroaryl”, as used herein, refers to aromatic groups containing one or more heteroatoms (O, S, or N). A heteroaryl group can be monocyclic or polycyclic. The heteroaryl groups of this invention can also include ring systems substituted with one or more oxo moieties. Examples of heteroaryl groups include, but are not limited to, pyridinyl, pyridazinal, imidaxolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotirazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furophridinyl, pyrolopyrimidinyl, and azaindoyl.

The foregoing heteroaryl groups may be C-attached or N-attached (where such is possible). For instance, a group derived from pyrrole may be pyrrol-1-yl (N-attached) or pyrrol-3-yl (C-attached).

In the context of the present invention, a bicyclic carbocyclic group is a bicyclic compound holding carbon only as a ring atom. The ring structure may in particular be aromatic, saturated, or partially saturated. Examples of such compounds include, but are not limited to, indanyl, naphthalenyl or azulenyl.

In the context of the present invention, an amino group may be primary (—NH₂), secondary (—NHRₐ), or tertiary (—NRₐR_{b}), wherein Rₐ and R_{b} may be: alkyl, alkenyl, alkynyl, alkoxy, cycloalkyl, cycloalkoxy, aryl, heteroaryl, or a bicyclic carbocyclic group.

The route of administration of the pharmacological agents of the present invention depends on the condition to be treated. The route of administration and the dosage to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual subject, and the severity of the subject's symptoms.

Compositions useful for the present invention can be administered parenterally such as, for example, by intravenous, intramuscular, intrathecal, or subcutaneous injection. Parenteral administration can be accomplished by incorporating the drug into a solution or suspension. Such solutions or suspensions may also include sterile diluents such as water for injection, saline solution, fixed oils, polyethylene glycols, glycerine, propylene glycol, or other synthetic solvents. Parenteral formulations may also include antibacterial agents such as, for example, benzyl alcohol, or methyl parabens, antioxidants, such as, for example, ascorbic acid or sodium bisulfite and chelating agents such as EDTA. Buffers such as acetates, citrates, or phosphates and agents for the adjustment of tonicity such as sodium chloride or dextrose may also be added. The parenteral preparation can be enclosed in ampules, disposable syringes, or multiple dose vials made of glass or plastic.

Rectal administration includes administering the pharmaceutical compositions into the rectum or large intestine. This can be accomplished using suppositories or enemas. Suppository formulations can be made by methods known in the art. For example, suppository formulations can be prepared by heating glycerin to about 120° C., dissolving the drug in the glycerin, mixing the heated glycerin after which purified water may be added, and pouring the hot mixture into a suppository mold.

Transdermal administration includes percutaneous absorption of the drug through the skin. Transdermal formulations include patches, ointments, creams, gels, salves, and the like. In a preferred embodiment the cholinergic agonist, nicotine, is administered transdermally by means of a nicotine patch.

A transesophageal device includes a device deposited on the surface of the esophagus which allows the drug contained within the device to diffuse into the blood which perfuses the esophageal tissue.

The present invention includes nasally administering to the subject an effective amount of the drug. As used herein, nasal administration includes administering the drug to the mucous membranes of the nasal passage or nasal cavity of the subject. As used herein, pharmaceutical compositions for nasal administration of a drug include effective amounts of the drug prepared by well-known methods to be administered, for example, as a nasal spray, nasal drop, suspension, gel, ointment, cream, or powder. Administration of the drug may also take place using a nasal tampon, or nasal sponge.

Accordingly, drug compositions designed for oral, lingual, sublingual, buccal, and intrabuccal administration can be used with the disclosed methods and made without undue experimentation by means well known in the art, for example, with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums, and the like.

Tablets, pills, capsules, troches, and the like may also contain binders, recipients, disintegrating agent, lubricants, sweetening agents, and flavoring agents. Some examples of binders include microcrystalline cellulose, gum tragacanth, or gelatin. Examples of excipients include starch or lactose. Some examples of disintegrating agents include alginic acid, corn starch, and the like. Examples of lubricants include magnesium stearate or potassium stearate. An example of a glidant is colloidal silicon dioxide. Some examples of sweetening agents include sucrose, saccharin, and the like. Examples of flavoring agents include peppermint, methyl salicylate, orange flavoring, and the like. Materials used in preparing these various compositions should be pharmaceutically pure and nontoxic in the amounts used.

Muscarinic agonists, can be administered orally, parenterally, intranasally, vaginally, rectally, lingually, sublingually, buccaly, intrabuccaly, or transdermally to the subject as described above, provided the muscarinic agonist can cross the blood-brain barrier or permeate the brain through circumventricular organs which do not have a blood brain barrier. Brain muscarinic agonists can also be administered by intracerebroventricular injection. NSAIDs, amiodarone, and αMSH may also be administered by intracerebroventricular injection or by one of the techniques described above, provided that they can permeate the brain through the blood-brain barrier or through circumventricular organs which do not have a blood brain barrier.

An effective amount is defined herein as a therapeutically or prophylactically sufficient amount of the drug to achieve the desired biological effect, here, treatment of ileus or alleviation of symptoms of ileus in a subject. Examples of effective amounts typically range from about 0.5 g/25 g body weight to about 0.0001 ng/25 g body weight, and preferably about 5 mg/25 g body to about 1 ng/25 g body weight.

The methods of the present invention can be used to treat ileus caused by abdominal surgery, or administration of narcotics or chemotherapeutic agents such as during cancer chemotherapy. Successful treatment of ileus includes reduction and alleviation of sumptoms of ileus. The terms “reduction” or “alleviation” when referring to symptoms of ileus in a subject, encompass reduction in measurable indicia over non-treated controls. Such measurable indicia include, but are not limited to retention time of gastric content after gavage and myeloperoxidase activity (units per gram) in the ileal musculature. In preferred embodiments, the measurable indicia are reduced by at least 20% over non-treated controls; in more preferred embodiments, the reduction is at least 70%; and in still more preferred embodiments, the reduction is at least 80%. In a most preferred embodiment, the symptoms of ileus are substantially eliminated.

As used herein, “treatment” includes pre-operative, peri-operative and post-operative treatment of ileus. Thus, “treatment” means prophylactic treatment of subjects at risk for ileus, for example, a subject undergoing abdominal surgery, experiencing abdominal surgery, or being administered narcotics or chemotherapeutic agents. The methods of the present invention can be used to treat ileus at the time of onset, and are also suitable for prophylactic treatment of ileus. “Prophylactic treatment” refers to treatment before onset of ileus to prevent, inhibit or reduce the occurrence of ileus. For example, a subject at risk for ileus, such as a subject undergoing abdominal surgery, or about to undergo abdominal surgery, or being (or about to be) administered narcotics or chemotherapeutic agents can be prophylactically treated according to the method of the present invention prior to the anticipated onset of ileus (for example, prior to, during, an/or for up to about 48 hours after abdominal surgery, prior to or during administration of narcotics or chemotherapeutics, but prior to the onset of ileus). “Treatment” also means therapeutic treatment, where the subject is already experiencing ileus.

While this invention has been particularly shown and described with references to example embodiments thereof, it will be understood by those skilled in the art that various changes in form and details may be made therein without departing from the scope of the invention encompassed by the appended claims.

What is claimed is:

1. A method of treating ileus in a subject, comprising administering to the subject an effective amount of a cholinergic agonist, wherein the cholinergic agonist is selective for an α7 nicotinic receptor.

2. The method of claim 1 wherein the α7 selective nicotinic agonist is a compound of structural formula III: 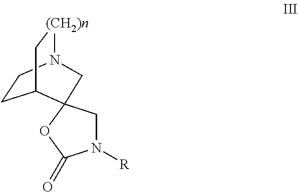
wherein:
R is hydrogen or methyl; and
n is 0 or 1; or
a pharmaceutically acceptable salt thereof.

3. The method of claim 1 wherein the α7 selective nicotinic agonist is (−)-spiro-[1-azabicyclo[2.2.2]octane-3,5′-octane-3,5′oxazolidin-2′-one.

4. The method of claim 1 wherein the α7 selective nicotinic agonist is a compound of structural formula IV: 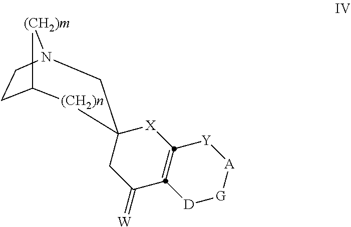
wherein:
m is 1 or 2;
n is 0 or 1;
Y is CH, N, or NO;
X is oxygen or sulfur;
W is oxygen, H₂, or F₂;
A is N or C(R²);
G is N or C(R³);
D is N or C(R⁴);
with the proviso that no more than one of A, G, and D is nitrogen but at least one of Y, A, G, and D is nitrogen or NO;
R¹ is hydrogen or C₁ to C₄ alkyl;
R², R³, and R⁴ are independently hydrogen, halogen, C₁-C₄ alkyl, C₂-C₄ alkenyl, C₂-C₄ alkynyl, aryl, heteroaryl, OH, OC₁-C₄ alkyl, CO₂R¹, —CN, —NO₂, —NR⁵, R⁶, —CF₃ or —OSO₂CF₃; or
R² and R³, or R³ and R⁴, respectively, may together form another six membered aromatic or heteroaromatic ring sharing A and G, or G and D, respectively, containing between zero and two nitrogen atoms, and substituted with one to two of the following substitutents: independently hydrogen, halogen, C₁-C₄ alkyl, C₂-C₄ alkenyl, C₂-C₄ alkynyl, aryl, heteroaryl, OH, OC₁-C₄ alkyl, CO₂R¹, —CN, —NO₂, —NR⁵R⁶, —CF₃ or —OSO₂CF₃;
R⁵ and R⁶ are independently hydrogen, C₁-C₄ alkyl, C(O)R⁷, C(O)NHR⁸, C(O)OR⁹, SO₂R¹⁰ or may together be (CH₂)ⱼQ(CH₂)ₖ; where Q is O, S, NR¹¹, or a bond;
j is 2 to 7;
k is 0 to 2; and
R⁷, R⁸, R⁹, R¹⁰, and R¹¹ are independently C₁-C₄, alkyl, aryl, or heteroaryl, an enantiomer thereof, or a pharmaceutically acceptable salt thereof.

5. The method of claim 4 wherein the α7 selective nicotinic agonist is (R)-(−)-5′-phenylspiro[1-azabicyclo[2.2.2]octane-3,2′octane-3,2′(3′H)-furo[2,3-b]pyridine].

6. The method of claim 1 wherein the α7 selective nicotinic agonist is a compound of structural formula V: 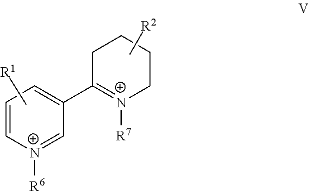
wherein:
R¹ is hydrogen or C₁-C₄ alkyl, R⁶, and R⁷ are independently selected from hydrogen, or C₁-C₄ alkyl or may be absent; and
R² is: 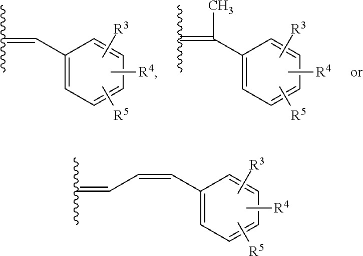
wherein:
R³, R⁴ and R⁵ are independently hydrogen, C₁-C₄ alkyl optionally substituted with N,N-dialkylamino having 1 to 4 carbons in each of the alkyls, C₁-C₆ alkoxy optionally substituted with N,N-dialkylamino having 1 to 4 carbons in each of the alkyls, carboalkoxy having 1 to 4 carbons in the alkoxy, amino, amido having 1 to 4 carbons in the acyl, cyano, and N,N-dialkylamino having 1 to 4 carbons in each of the alkyls, halo, hydroxyl, or nitro.

7. The method of claim 6 wherein the α7 selective nicotinic agonist is 3-(4-hydroxy-2-methoxybenzylidene)anabaseine.

8. The method of claim 6 wherein the α7 selective nicotinic agonist is 3-(2,4-dimethoxybenzylidene)anabaseine (DMXB-A).

9. The method of claim 2 wherein the α7 selective nicotinic agonist is (1-aza-bicyclo[2.2.2]oct-3-yl)-carbamic acid 1-(2-fluorophenyl)-ethyl ester.

10. The method of claim 2 wherein the α7 selective nicotinic agonist is cocaine methiodide.

11. The method of claim 2 wherein the α7 selective nicotinic agonist is choline.

12. A method of treating ileus in a subject, comprising administering to the subject an effective amount of a cholinergic agonist, wherein the α7 selective nicotinic agonist is a compound of structural formula VI: 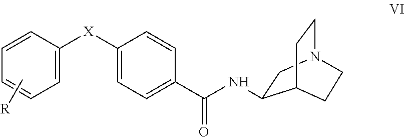
wherein:
X is O or S;
R is H, OR¹, NHC(O)R¹, or a halogen; and
R¹ is a C₁-C₄ alkyl; or
a pharmaceutically acceptable salt thereof.

13. A method of treating ileus in a subject, comprising administering to the subject an effective amount of a cholinergic agonist, wherein the cholinergic agonist is administered via a transdermal or trans-esophogeal device.
